(19) United States
(12) Patent Application Publication
(10) Pub. No.: US 2025/0121238 A1
(43) Pub. Date: Apr. 17, 2025

(54) PFAS CHEMICAL DESTRUCTION PROCESS

(71) Applicant: PERMA-FIX ENVIRONMENTAL SERVICES, INC., Atlanta, GA (US)

(72) Inventors: Louis F. Centofanti, Atlanta, GA (US); Tommy W. Yarbrough, Atlanta, GA (US); Jose Gabriel Garcia, Atlanta, GA (US); Christopher M. Dyer, Atlanta, GA (US)

(21) Appl. No.: 18/888,283

(22) Filed: Sep. 18, 2024

Related U.S. Application Data
(60) Provisional application No. 63/655,844, filed on Jun. 4, 2024.
(60) Provisional application No. 63/555,113, filed on Feb. 19, 2024.
(60) Provisional application No. 63/602,736, filed on Nov. 27, 2023.
(60) Provisional application No. 63/544,544, filed on Oct. 17, 2023.

(51) Int. Cl. A62D 3/34; A62D 3/36; A62D 3/40; A62D 101/22; C07C 1/30

(52) U.S. Cl. CPC A62D 3/34; A62D 3/36; A62D 3/40; C07C 1/30; A62D 2101/22

(57) ABSTRACT

A system and method for chemically destroying, degrading and incinerating a fluorocarbon or fluorinated material, such as perfluoroalkyl and polyfluoroalkyl substances (PFAS), with reduced emissions of gaseous PFC is provided. The method includes mixing the fluorinated material, a hydroxide base, and optionally a solvent system in a batch reactor to form a suspension. The PFAS and solvent system can be provided by AFFF. The reaction mixture is heated to a temperature ranging from about 25° C. to about 400° C. for about 0.5 hours to about 240 hours to defluorinate the fluorocarbons in the PFAS and produce a defluorinated waste product. More specifically, the method converts organic fluorine present in the PFAS to inorganic fluoride. Thus, the defluorinated waste product can be incinerated with reduced emissions of harmful gaseous PFCs.

### CROSS-REFERENCE TO RELATED APPLICATIONS

This U.S. non-provisional patent applications claims the benefit of and priority to U.S. provisional patent application Ser. No. 63/544,544, filed Oct. 17, 2023; U.S. provisional patent application Ser. No. 63/602,736, filed Nov. 27, 2023; U.S. provisional patent application Ser. No. 63/555,113, filed Feb. 9, 2024; and U.S. provisional patent application Ser. No. 63/655,844, filed Jun. 4, 2024, the entire disclosures of which are hereby incorporated by reference in their entirety.

### BACKGROUND

### 1. Field of the Invention

The current invention relates generally to a system and method for chemically destroying and disposing of fluorocarbon or fluorinated materials, for example perfluoroalkyl and polyfluoroalkyl substances (PFAS).

### 2. Related Art

Fluorocarbons and fluorinated substances, such as per- and polyfluoroalkyl substances (PFAS), are anthropogenic synthetic materials used for over 90 years (1) and found in water at harmful concentrations to humans (2). PFAS have been designated as emerging contaminants of concern since 2000 (3). They can bioaccumulate in humans (4, 5, 6), have been linked to certain cancers, and have a wide range of deleterious effects including hormone and immune system interferences, ulcerative colitis, and endocrine disruption (7, 8 9), to name a few.

PFAS compose a diverse class of chemicals that, due to their low surface tension and wetting properties, are found in a wide range of products and processes, including fluoropolymers, liquid repellants for paper, packaging, textile, leather, and carpet goods, industrial surfactants, additives, coatings, and firefighting foams. (10) Fluoropolymers, for example polytetrafluoroethylene (PTFE), are believed to be the most commonly found PFAS for computer applications and surface coatings.

The United States Environmental Protection Agency (USEPA) CompTox database has identified over 9000 highly fluorinated substances with Chemical Abstracts Service numbers available in the global market, the majority being fluorinated polymers and fluorinated surfactants (11).

The most recent guideline effective on Jun. 25, 2024. has advisory limits of 4 parts per trillion (ppt) of PFOA, (12, 13) and PFOS, the USEPA added five more PFAS compounds for site cleanups (perfluoronanoic acid (PFNA), PFHxS, perfluorononanoate, perfluorooctanoate, and perfluorohexanesulfonate) based on risk-based values for regional screening levels (RSLs) (3). Various techniques have been used to decompose or dispose of hazardous substances, such as PFAS by electrochemical oxidation, direct irradiation, plasma treatment, photocatalysis, sonolysis, supercritical water oxidation, reductive hydrodefluorination and thermal degradation/incineration (14).

In 2020, the U.S. EPA (15) published a technical brief on the incineration of PFAS with the main conclusion that the effectiveness of incineration in destroying PFAS and their fate in terms of potential mixed fluorinated organic byproduct formation is not clearly understood. A significant concern is that incomplete destruction of PFAS can result in the formation of PIC (products of incomplete combustion), e.g., smaller PFAS molecules, which could be a potential hazard. Only a few studies are available related to PFAS incineration in full-scale operating facilities (16, 17-19). According to Solo-Gabriele et al., increasing incinerator temperatures decreased the total treated PFAS concentrations. However, not all PFAS species decreased with increasing temperatures (17). There is an alarming report of higher concentrations of PFOA found in the air at the incinerator sites compared to upwind sites (18). Public concern is that the incineration may spread PFAS and not break them down. This publication claims that the preliminary data show that soil and surface water near a commercial facility in Cohoes, New York, that has burned firefighting foam containing PFAS are contaminated with PFAS (20).

PFAS incineration can occur directly for PFAS-based materials, such as firefighting foams or indirectly via the incineration of waste containing PFAS, such as textiles, etc. (21). Recently the Defense Department issued a ban on incinerating PFAS-laden items, with particular emphasis on the aqueous film-forming foam often used in training and combat situations (22). In addition, under the 2022 National Defense Authorization Act (23), the military is now prohibited from incinerating PFAS-containing materials in accordance with the Clean Air Act (24). Most incineration studies monitored a limited number of compounds, leaving the question of “unmonitored” PFAS unanswered (25). Even though multiple studies were done on the thermal degradation of PFAS (26, 27, 28, 29, 30), only limited data (31, 32) is available on directly detecting degradation products during field-scale incineration. The main obstacle is still the lack of both suitable emission sampling methods (including industrial field sampling) to capture PFAS compounds and analytical methods to identify/detect PFAS and their thermal decomposition byproducts. The question remains unanswered as to how significant is the portion of volatile species that escape the analysis.

Thermal degradation/incineration is the widely available approach for managing contaminated solids, liquids, or gases using already built incinerator facilities (33), and many incineration facilities are already knowingly or unknowingly treating PFAS (e.g., consumer products, activated carbon regeneration). As previously mentioned, incineration facilities have already been deployed and are well-established in the industry.

Therefore, the initial cost of implementing the technology will be significantly reduced compared to other PFAS-destructive technologies. Together, these advantages place them as a critical solution for managing PFAS-containing waste (34, 35).

However, in general, waste byproducts in any incineration include bottom ash, which contains non-combusted products, and gas, containing tiny particles and volatile products (34). According to Wang et al. (34) regarding PFAS incineration, the resulting ash and gas are both problematic. Ash contains inorganic fluorine and remaining PFAS bound to inorganic compounds such as calcium. Ash is typically sent to a landfill or repurposed. Particulates in the gas can be captured with electrostatic precipitators. However, HF is anticipated to be the main product of PFAS thermal conversion during incineration and is a corrosive/acidic gas. Capturing or removing volatile fluoride-containing byproducts may also be problematic.

Any untreated PFAS or byproducts from incineration are released directly into the environment (34). Therefore, the potential risk of secondary air and soil pollution and the return of PFAS into the environment is very high. In addition, incomplete destruction during thermal treatment/incineration could generate an unknown array of byproducts, which might be environmentally problematic. Since current knowledge of the fate of PFAS is limited, there is concern that PFAS incineration can release toxic gases (tetrafluoromethane, hexafluoroethane, fluoro-dioxins, fluoro-benzofurans, and perfluorinated carboxylic acids) (36, 37).

Thermal treatment, such as incineration, is a popular and effective technique used to dispose of hazardous substances in land-scarce and resource-lacking locations because it can greatly reduce volume of the waste and can produce electricity. However, a significant concern associated with incineration of the waste is emission of harmful gases. Dioxin and furan are commonly studied gases because they are serious health hazards. There are few studies on the production of perfluorinated compound (PFC) gases from the thermal treatment process. However, it was reported that thermal decomposition of PFAS can form gas-phase PFCs, such as CF₄ and C₂F₆, which are harmful to the environment. Gaseous PFCs are potent greenhouse gases. The global warming potential of CF₄ is 6500 times that of CO2, and the atmospheric lifetime of C₂F₆ is 50,000 years. Due to the long atmospheric lifetime of gaseous PFCs, gaseous PFC emissions can permanently alter the radiative budget of the atmosphere. Other methods for disposing PFAS are being investigated, but fluorinated halogenated hydrocarbons, such as those found in PFAS, are very difficult to destroy due to the strength of the carbon-fluorine bond. There remains a need for a more sustainable method for disposing of PFAS.

### SUMMARY

One aspect of the invention provides a method for chemically destroying and disposing a fluorinated material. The method comprising the steps of: placing a fluorinated material, a hydroxide base, and optionally a solvent system in a batch reactor to form a suspension, and heating the suspension in the batch reactor to produce a defluorinated waste product. The solvent system (“solvent”) includes at least one of a diglyme, polyether, polyether alcohol, a polyethylene glycol selected from ethylene glycol and one or more polyethylene glycol referred to as PEG50 through PEG3350, N-methylpyrrolidine, cyrene and/or water.

The fluorinated material is typically a per- and/or a polyfluoroalkyl substance (PFAS).

The method can include placing an aqueous film-forming foam (AFFF) in the batch reactor, wherein the AFFF contains the PFAS and optionally the “solvent”.

According to an example embodiment, the fluorinated material, the hydroxide base, and optionally the “solvent” are placed in the batch reactor and allowed to react with one another for a time of about 0.5 hours to about 240 hours; and the heating step includes heating the suspension to a temperature ranging from about 25° C. to about 400° C.

According to example embodiments, the hydroxide base includes at least one of potassium hydroxide (KOH), calcium hydroxide (Ca(OH)₂), cesium hydroxide (CsOH), lithium hydroxide (LiOH), sodium hydroxide (NaOH), strontium hydroxide (Sr(OH)₂) and mixtures thereof.

According to example embodiments, the “solvent” can be added to the batch reactor and includes at least one of selected from diglyme, polyethers, polyether alcohol, a polyethylene glycol selected from ethylene glycol and PEG50 through PEG3350, N-methylpyrrolidine, cyrene or water.

According to example embodiments, the hydroxide base includes a mixture of two hydroxide bases, and the hydroxide bases are in a ratio of about 1:99 w/w % to about 99:1 w/w %, for example a ratio of about 25:75 w/w % to about 75:25 w/w %.

The solvent and the fluorinated material can be provided to the batch reactor by a single substance, and no additional solvent needs to be added.

According to example embodiments, the fluorinated material, the hydroxide base, and the “solvent” are placed in the batch reactor and allowed to react with one another for a time of about 0.5 hours to about 240 hours, or about 3 hours to about 120 hours, or about 4 hours to about 60 hours, or about 4 hours to about 10 hours.

The heating step typically includes heating the suspension to a temperature of about 25° C. to about 300° C., or about 150° C. to about 200° C.

According to an example embodiment, the hydroxide base is potassium hydroxide; the heating step includes heating to a temperature of about 180° C.; the fluorinated material is PFAS; the PFAS, the hydroxide base, and the solvent are placed in the batch reactor and allowed to react with one another for a time of about 4 hours to about 8 hours; the PFAS and the solvent are provided by a single substance; and no additional solvent is added to the batch reactors.

The method can further include incinerating the defluorinated waste product.

Another aspect of the disclosure provides a system comprising a batch reactor for chemically destroying a fluorinated material according to the method described herein.

Another aspect of the disclosure provides a method for chemically destroying and disposing PFAS which comprises placing a fluorinated material, a hydroxide base, and optionally a “solvent” in a batch reactor to form a suspension; and maintaining the fluorinated material in the batch reactor until a defluorinated waste product is produced. The fluorinated material can be maintained in the batch reactor at room temperature.

### BRIEF DESCRIPTION OF THE DRAWING

Other advantages of the present invention will be readily appreciated, as the same becomes better understood by reference to the following detailed description when considered in connection with the accompanying drawing wherein:

FIG. 1 illustrates a batch system for defluorinating, chemically destroying a fluorocarbon present in PFAS to produce a defluorinated waste product according to an example embodiment.

### DETAILED DESCRIPTION OF EXAMPLE EMBODIMENTS

The materials, compounds, compositions, and methods described herein may be understood more readily by reference to the following detailed description of specific aspects of the disclosed subject matter and the examples included therein.

It is to be understood that the terminology used herein is for the purpose of describing only and is not intended to be limiting. Also, throughout this specification, various publications are referenced. The disclosures of these publications are hereby incorporated by reference into this application in order to more fully describe the state of the art to which the disclosed matter pertains. The references disclosed are also individually and specifically incorporated by reference herein for the material contained in them.

In this specification and the claims that follow, reference will be made to several terms, which shall be defined to have the following meanings:

Throughout the specification and claims the word “comprise” and other forms of the word, such as “comprising” and “comprises,” means including but not limited to, and is not intended to exclude, for example, other additives, solvents, bases, components, integers, or steps. As used herein, the singular forms “a,” “an,” and “the” include singular and plural referents unless the context clearly dictates otherwise. Thus, for example, reference to “a composition” includes mixtures of two or more such compositions. “Optional” or “optionally” means that the subsequently described event or circumstance can or cannot occur, and that the description includes instances where the event or circumstance occurs and instances where it does not. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Unless stated otherwise, the term “about” means within 5% (e.g., within 2% or 1%) of the particular value modified by the term “about.”

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms. As used herein, the term “composition” is intended to encompass a product comprising the specified ingredients in the specified amounts, as well as any product which results, directly or indirectly, from combination of the specified ingredients in the specified amounts. References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight component Y, X and Y are present at a weight ratio of 2:5 and are present in such ratio regardless of whether additional components are contained in the mixture. A weight percent (wt. %) of a component, unless specifically stated to the contrary, is based on the total weight of the formulation or composition in which the component is included. As used herein, the term “substituted” is contemplated to include all permissible substituents of inorganic base compounds. In a broad aspect, the permissible substituents include all alkali and alkaline-earth metals in the periodic table. Illustrative substituents include, for example, those described below. The permissible substituents can be one or more and the same or different for appropriate inorganic base compounds.

Those persons of ordinary skill in the art will appreciate that Compounds of Formula I are examples of inorganic base analogs. As used herein, “an analog of potassium hydroxide” or “analogs of potassium hydroxide” are not limited to those analog compounds represented by Formula I, and may include many additions or substitutions of elements, groups, or moieties to the chemical structure of potassium hydroxide.

M(OH)x Formula I
wherein
M is selected from the alkali or alkaline-earth metal groups; and,
x is the number of hydroxy units per M valence;

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

One aspect of the invention provides a system and method for disposing of per- and/or polyfluoroalkyl substances (PFAS) with reduced emissions of gaseous PFC, such as CF₄ and C₂F₆. In certain embodiments, the PFAS is a single per- and/or polyfluorinated compound or a mixture of several per- and/or polyfluorinated compounds. The current invention also pertains to a method of adding a solvent to the PFAS and applying several heating temperatures in the degradation process. More specifically, the subject matter disclosed herein relates to a system and method that can be used for reducing emissions of gaseous perfluorinated compounds (PFCs) during thermal treatment of PFAS.

Various types of PFAS can be treated with the batch system according to the present invention, for example perfluorooctanoic acid (PFOA). Although the system and method are typically applied to PFAS, and PFAS will be discussed throughout the present disclosure, the system and method can be used to dispose of any type of fluorocarbon or fluorinated material. The system and method destroy the carbon-fluorine bonds and converts the organic fluorine present in the fluorocarbon or other fluorinated material to inorganic fluoride.

The method for chemically destroying and disposing the PFAS first includes placing the PFAS in a batch system, more specifically in a batch reactor. The PFAS is added in the batch reactor along with a hydroxide base, for example potassium hydroxide (KOH), calcium hydroxide (Ca(OH)₂), cesium hydroxide (CsOH), lithium hydroxide (LiOH), strontium hydroxide (Sr(OH)₂) and/or sodium hydroxide (NaOH). A “solvent” belonging to the general class of diglyme, polyethers, polyether alcohol, a polyethylene glycol selected from ethylene glycol and PEG50 through PEG3350, N-methylpyrrolidine, cyrene and/or water is also optionally added to the batch reactor to form a solution. Water may also be present optionally as a co-solvent in the batch reactor. The PFAS may be provided in an aqueous film-forming foam (AFFF), also known as a firefighting foam. The AFFF is typically a suspension composition. Also, some of the “solvent” listed above may already be present in the AFFF suspension composition.

The PFAS is typically maintained in the batch reactor at a temperature of ranging from room temperature for several days, or 100° C. and 200° C. for at least 2 hours, for example 3 to 5 hours, or up to 8 hours to chemically destroy or defluorinated the PFAS and produce a defluorinated waste product consisting of inorganic fluoride. Some types of PFAS, such as perfluorooctyl sulfonate (PFOS), may require higher temperatures and longer times in the reactor, for example temperatures up to but not limited to 300° C. According to other embodiments, the temperature of the batch system may be less than 100° C., for example room temperature or 50° C. up to 100° C. When the temperature of the batch system is lower, the time required to defluorinate the PFAS—and produce a defluorinated waste product consisting of an inorganic fluoride is longer. The defluorinated waste product produced may typically include polyethylene glycol and/or the solvent used in the reactor, formate, carbonate, oxalate and/or glycolate, and inorganic fluoride(s) wherein the composition of the inorganic fluoride, i.e. potassium fluoride, sodium fluoride, lithium fluoride, strontium fluoride and/or calcium fluoride or combinations thereof, etc., depends on the hydroxide base or mixture of hydroxide bases used in the batch system, The defluorinated waste product can be further incinerated without significant emissions of the harmful gaseous PFCs.

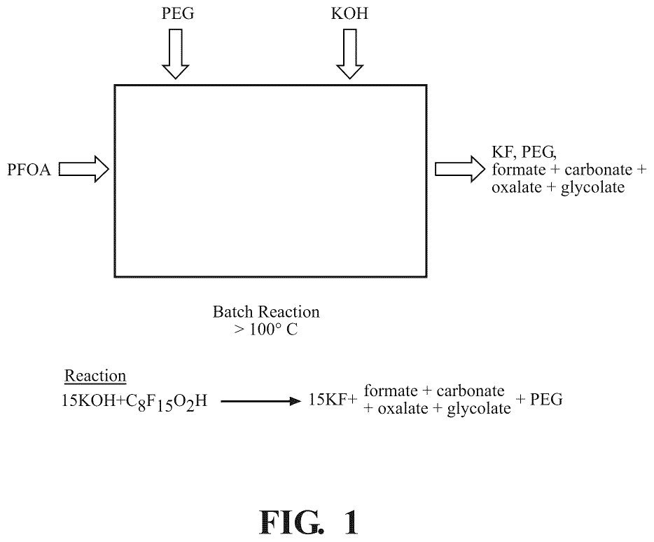

FIG. 1 illustrates the batch system and method for chemically destroying or defluorinating a fluorocarbon present in PFAS to produce a defluorinated waste product according to an example embodiment. According to this embodiment, the following reaction occurs in the batch example system:

15KOH+C₈F₁₅O₂H→15KF+formate+carbonate+oxalate+glycolate.

According to this example, PFAS is placed in the batch reactor along with PEG and potassium hydroxide (KOH). The PEG is preferably PEG200 which has a molar mass of 190-210 g/mol and a chemical formula of H—(O—CH₂CH₂)ₙ—OH, where n=8.2 to 9.1. It is believed that the PEG200 could be replaced optionally with diglyme, polyethers, polyether alcohol, a polyethylene glycol selected from ethylene glycol and PEG50 through PEG3350, N-methylpyrrolidine, cyrene and/or water. Alternatively, no solvent is added since some of these solvents may already be present in the AFFF suspension composition, and the KOH could be replaced with another hydroxide base comprised of but not limited to sodium, calcium, lithium, strontium or cesium or optionally mixtures thereof, etc. According to this example, the PFAS is allowed to react in the batch system at a temperature of 180° C. to 200° C. for approximately 4 hours at ambient pressure. The resulting defluorinated waste product includes the product generated potassium fluoride (KF), PEG200, unreacted excess potassium hydroxide (KOH), and carbonate and/or formate and/or oxalate and/or glycolate or mixtures thereof.

After the batch process, the defluorinated waste product can be thermally treated, for example by incineration, with reduced emissions of the hazardous gaseous PFCs, such as CF₄ and C₂F₆.

Before incineration, some of the components present in the defluorinated waste product can be recycled or removed and disposed of without thermal treatment. For example, according to one embodiment, the PEG200 is removed from the defluorinated waste product and recycled. The recycled PEG200 can be used in future batch systems.

Another aspect of the invention is the capability of reusing the unreacted components in the process of defluorination of the AFFF suspension substances. Upon reaction completion, the resulting mixture is treated with caustic lime (calcium hydroxide) which reacts with the inorganic fluoride species that are produced during the AFFF suspension components defluorination reaction. The resulting calcium fluoride from the caustic lime treatment precipitates out of the entire combination of components. Upon filtration of the precipitated calcium fluoride, the filtrate of the reaction mixture can then be reused in a subsequent process.

### EXPERIMENT

### General Procedure for the Defluorination Reaction.

AFFF suspension was treated with a hydroxide base (sodium hydroxide, potassium hydroxide or calcium hydroxide or strontium hydroxide or combinations thereof in various ratios) either neat or in the presence of a “solvent” in various ratios at 150° C. to 200° C. for 4 hours. The resulting reaction mixture is allowed to cool to room temperature then analyzed by 19F NMR.

### Representative Example 1. “Solvent” Assisted

In a 40 mL vial with a screwcap, PEG200 (3 equivalents w/w, 6 g) is added to crushed potassium hydroxide pellets (0.75 equivalent w/w, 1.5 grams) followed by the addition of AFFF (PFAS) suspension (1 equivalent w/w, 2 grams). The vial is immersed in a pre-heated sand bath (hot plate T: 150° C. to 200° C.) and allowed to react for 4 hours. The resulting amber colored reaction mixture is allowed to cool to room temperature. A small blob particulate is formed which is separated from the reaction mixture by sonication for 15 minutes followed by centrifugation at 3000 rpm for 15 minutes and decanting. Both the decantate and the residue are analyzed by ¹⁹F NMR. The reaction mixture shows the presence of only inorganic potassium fluoride.

### Representative Example 2. Neat

In a 40 mL vial with a screwcap, AFFF (PFAS) suspension (1 equivalent w/w, 2 grams) is added to crushed potassium hydroxide pellets (0.75 equivalent w/w, 1.5 grams). The vial is immersed in a pre-heated sand bath (hot plate T: 150° C. to 200° C.) and allowed to react for 4 hours. The resulting reaction mixture is allowed to cool to room temperature followed by ¹⁹F NMR analysis.

A small blob particulate is formed which is separated from the reaction mixture by sonication for 15 minutes followed by centrifugation at 3000 rpm for 15 minutes and decanting. Both the decantate and the residue are analyzed by ¹⁹F NMR. The reaction mixture shows the presence of only inorganic potassium fluoride.

An experiment was conducted to confirm that the batch system and method disclosed herein can successfully destroy PFOA and can convert the organic fluorine present in the PFOA to innocuous inorganic fluoride.

The experiment included dissolving reagent grade perfluorooctanoic acid (PFOA—CAS 335-67-1) in PEG200 crushed potassium hydroxide pellets (KOH—CAS 1310-58-3) is added to produce a solution containing approximately 67% PEG200 and 33% KOH by weight. This 5000 ppm PFOA solution was stirred and heated to approximately 150° C. to 200° C. for four hours in a closed 40-mL screwed cap vial.

The reaction mixture was then analyzed by ¹⁹Fluorine-Nuclear Magnetic Resonance (NMR). Before the reaction was initiated by heating, the NMR spectra showed peaks characteristic of the PFOA molecule. All PFOA peaks had disappeared and only a large fluoride peak remained. The ¹⁹F-NMR results provided a qualitative indication that PFOA was destroyed, and organic fluorine was converted into innocuous inorganic fluoride. These qualitative NMR results indicate effectiveness of the batch system and method disclosed herein should be at least 98%. LCMSMS analysis corroborated the qualitative results in addition to quantifying the unreacted PFOA to 0.006 ppm.

In summary, in accordance with the purposes of the disclosed materials and methods, as embodied and broadly described herein, the disclosed subject matter, in one aspect, relates to the composition and methods of defluorination of PFAS generating inorganic fluoride in the form of a salt. Moreover, it relates to methods of reducing emissions of gaseous perfluorinated compounds (PFCs) during thermal treatment of PFAS. In specific aspects, the disclosed subject matter relates to the selection of materials for a greener process.

Certain embodiments of this invention provide a composition comprising at least one “solvent” system and a strong base, wherein the hydroxide base is comprised of potassium hydroxide, sodium hydroxide, cesium hydroxide, lithium hydroxide, strontium hydroxide and/or calcium hydroxide either by themselves or in combination at different compositions in w/w % according to reaction scheme I.

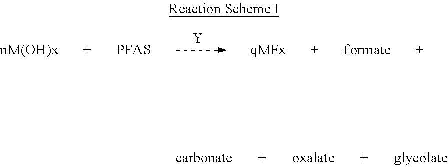

wherein;
n is the minimum amount of molar equivalents to degrade the organic fluorine in PFAS;
M is comprised of potassium, sodium, cesium, lithium, strontium and/or calcium;
q is the maximum amount of molar equivalents generated by degradation of the organic fluorine in PFAS;
x is the number of hydroxy (OH) and fluoride (F) units per M valence; and,
Y is comprised of a solvent from the possible “solvent” described above.

In another embodiment, these compositions as described hereinabove, do not include addition of said “solvent” according to the reaction scheme II.

nM(OH)x+PFAS→qMFx+formate+carbonate+oxalate+glycolate Reaction Scheme II
wherein;
n is the minimum amount of molar equivalents to degrade the organic fluorines in PFAS;
M is comprised of potassium, sodium, cesium, lithium, strontium and/or calcium;
q is the maximum amount of molar equivalents generated by degradation of the organic fluorine in PFAS; and,
x is the number of hydroxy (OH) and fluoride (F) units per M valence.

A system and method for chemically destroying and disposing per- and/or polyfluoroalkyl substances (PFAS) with reduced emissions of gaseous PFCs is provided. The PFAS is placed in a batch system containing a hydroxide base with or without the presence of a “solvent” to defluorinate the fluorocarbon(s) present in the PFAS mixture forming a defluorinated waste product such as formate, carbonate, oxalate, and glycolate. After defluorinating the fluorocarbon compound mixture of the PFAS, a thermal treatment, for example incineration, may be performed on the defluorinated waste product with reduced emissions of the harmful gaseous PFCs.

As indicated above, the system and method for treating and disposing fluorinated material, such as per- and/or polyfluoroalkyl substances (PFAS) with reduced emissions of gaseous PFCs can be applied to soils or other environmental media containing the fluorinated material.

Obviously, many modifications and variations of the present invention are possible in light of the above teachings and may be practiced otherwise than as specifically described while within the scope of the following disclosure and claims.

### LITERATURE CITED

1. Meegola, J. N. et al. Int. J. Environ. Res. Public Health 2022, 19 (24), 16397.
2. Ebnesajjad, S. Introduction to Fluoropolymers; Elsevier: Amsterdam, The Netherlands, 2013, 17-35.
3. Camdzic, D. et al. J. Hazard. Mater. Lett. 2021, 2, 100023.
https://www.sciencedirect.com/science/article/pii/S2666911021000113#bib0285
4. Arana Juve, J.-M. et al. Curr. Opin. Chem. Engin. 2023, 41, 100943.
5. Buck, R. C. et al. Integr. Environ. Assess. Manag., 2011, 7 (4), 513-541.
6. Ying, L. Occup. Environ. Med., 2018, 75 (1), 46-51.
7. Kelly, B. C. et al. Environ. Sci. Technol., 2009, 43 (11), 4037-4043.
8. Jiang, Q. et al. Toxicological Effects of Perfluoroalkyl and Polyfluoroalkyl Substances 2015, 177-201.
9. Abbott, B. D. et al. Reprod. Toxicol., 2012, 33 (4), 491-505.
10. Leeson, A. et al., Environ. Toxicol. Chem. 2021. 40, 24-36.
11. Wang, Z. et al., Environ. Int. 2013, 60, 242-8.
Vierke, L. et al., Environ. Sci. Eur. 2012, 24, 16.
Leung, S. C. E. et al., Sci. Total Environ. 2022, 827, 153669.
https://comptox.epa.gov/dashboard/chemical-lists/PFASSTRUCT;
https://comptox.epa.gov/dashboard/chemical-lists/PFASDEV
12. Krafft, M. P. et al. Curr. Opin. Colloid Interface Sci. 2015, 20, 192-212.
Ojo, A. F. et al. J. Hazard. Mater. 2021, 407, 12486
13. https://www.whitehouse.gov/wp-content/uploads/2023/03/OSTP-March-2023-PFAS-Report.pdf
https://www.epa.gov/sdwa/and-polyfluoroalkyl-substances-pfas
14. Int. J. Environ. Res. Public Health 2022, 19 (24), 16397
15. EPA. Per- and Polyfluoroalkyl Substances (PFAS): Incineration to Manage PFAS Waste Streams Background. US EPA Tech. Brief. 2020.
16. Water Res. 2007, 41, 4611-4620
17. Waste Manag. 2020, 107, 191-200
18. Sci. Total Environ. 2020, 705, 135832.
19. Water Environ. Res. 2021, 93, 826-843
20. https://cen.acs.org/environment/persistent-pollutants/Incincerators-spread-break-down-PFAS/98/web/2020/04
21. Stoiber, T.; Evans, S.; Naidenko, O. V. Disposal of products and materials containing per- and polyfluoroalkyl substances (PFAS): A cyclical problem. Chemosphere 2020, 260, 127659.
22. Crunden, E. A. Defense Department Hits the Brakes on PFAS Incineration. Available online: https://www.eenews.net/articles/defense-department-hits-the-brakes-on-pfas-incineration/.
23. U.S.C. H.R.2591 PFAS Waste Incineration Ban Act of 2019. 2019. Available online: https://www.govinfo.gov/
24. U.S.C. Public Law 116-92-National Defense Authorization Act for Fiscal Year 2020. 2019. Available online: https://www.govinfo.gov/
25. Horst, J.; McDonough, J.; Ross, I.; Houtz, E. Understanding and managing the potential byproducts of PFAS destruction. Groundw. Monit. Remediat. 2020, 40, 17-27.
26. Stockenhuber, S.; Weber, N.; Dixon, L.; Lucas, J.; Grimison, C.; Bennett, M.; Stockenhuber, M.; Mackie, J.; Kennedy, E. Thermal Degradation of Perfluorooctanoic Acid (PFOA). In Proceedings of the 16th International Conference on Environmental Science and Technology, Rhodes, Greece, 4-7 Sep. 2019.
27. Altarawneh, M.; Almatarneh, M. H.; Dlugogorski, B. Z. Thermal decomposition of perfluorinated carboxylic acids: Kinetic model and theoretical requirements for PFAS incineration. Chemosphere 2022, 286, 131685.
28. Sasi, P. C.; Alinezhad, A.; Yao, B.; Kubátová, A.; Golovko, S. A.; Golovko, M. Y.; Xiao, F. Effect of granular activated carbon and other porous materials on thermal decomposition of per- and polyfluoroalkyl substances: Mechanisms and implications for water purification. Water Res. 2021, 200, 117271.
29. Xiao, F.; Sasi, P. C.; Yao, B.; Kubátová, A.; Golovko, S. A.; Golovko, M. Y.; Soli, D. Thermal decomposition of PFAS: Response to comment on “thermal stability and decomposition of perfluoroalkyl substances on spent granular activated carbon”. Environ. Sci. Technol. Lett. 2021, 8, 364-365.
30. Crownover, E.; Oberle, D.; Kluger, M.; Heron, G. Perfluoroalkyl and polyfluoroalkyl substances thermal desorption evaluation. Remediat. J. 2019, 29, 77-81.
31. Yamada, T.; Taylor, P. H.; Buck, R. C.; Kaiser, M. A.; Giraud, R. J. Thermal degradation of fluorotelomer treated articles and related materials. Chemosphere 2005, 61, 974-984.
32. Taylor, P. H.; Yamada, T.; Striebich, R. C.; Graham, J. L.; Giraud, R. J. Investigation of waste incineration of fluorotelomer-based polymers as a potential source of PFOA in the environment. Chemosphere 2014, 110, 17-22.
33. Vidonish, J. E.; Zygourakis, K.; Masiello, C. A.; Sabadell, G.; Alvarez, P. J. J. Thermal treatment of hydrocarbon-impacted soils: A review of technology innovation for sustainable remediation. Engineering 2016, 2, 426-437.
34. Wang, J.; Lin, Z.; He, X.; Song, M.; Westerhoff, P.; Doudrick, K.; Hanigan, D. Critical review of thermal decomposition of per- and polyfluoroalkyl substances: Mechanisms and implications for thermal treatment processes. Environ. Sci. Technol. 2022, 56, 5355-5370.
35. Aleksandrov, K.; Gehrmann, H. J.; Hauser, M.; Mätzing, H.; Pigeon, D.; Stapf, D.; Wexler, M. Waste incineration of polytetrafluoroethylene (PTFE) to evaluate potential formation of per- and poly-fluorinated alkyl substances (PFAS) in Flue Gas. Chemosphere 2019, 226, 898-906.
36. García, A. N.; Viciano, N.; Font, R. Products obtained in the fuel-rich combustion of PTFE at high temperature. J. Anal. Appl. Pyrolysis 2007, 80, 85-91.
37. Wang, F.; Shih, K.; Lu, X.; Liu, C. Mineralization behavior of fluorine in perfluorooctanesulfonate (PFOS) during thermal treatment of lime-conditioned sludge. Environ. Sci. Technol. 2013, 47, 2621-2627.

It will be appreciated by those persons skilled in the art that changes could be made to embodiments of the present invention described herein without departing from the broad inventive concept thereof. It is understood, therefore, that this invention is not limited by any particular embodiments disclosed but is intended to cover the modifications that are within the spirit and scope of the invention, as defined by the appended claims.

What is claimed is:

1. A method for chemically destroying and disposing a fluorinated material, comprising the steps of:
placing a fluorinated material, a hydroxide base, and optionally a solvent system in a batch reactor to form a suspension; and
heating the suspension in the batch reactor to produce a defluorinated waste product.

2. The method of claim 1, wherein the fluorinated material is a per- and/or polyfluoroalkyl substance (PFAS).

3. The method of claim 2 including placing an aqueous film-forming foam (AFFF) in the batch reactor, wherein the AFFF contains the PFAS and optionally the solvent.

4. The method of claim 1, wherein the fluorinated material, the hydroxide base, and optionally the solvent system are placed in the batch reactor and allowed to react with one another for a time of about 0.5 hours to about 240 hours; and the heating step includes heating the suspension to a temperature ranging from about 25° C. to about 400° C.

5. The method of claim 1, wherein the hydroxide base includes at least one of potassium hydroxide (KOH), calcium hydroxide (Ca(OH)₂), cesium hydroxide (CsOH), lithium hydroxide (LiOH), strontium hydroxide (Sr(OH)₂), sodium hydroxide (NaOH), and mixtures thereof.

6. The method of claim 1, wherein the solvent system is comprised of diglyme, polyethers, polyether alcohol, a polyethylene glycol selected from ethylene glycol and PEG50 through PEG3350, N-methylpyrrolidine, cyrene and/or water.

7. The method of claim 1, wherein the hydroxide base includes a mixture of two hydroxide bases, and the hydroxide bases are in a ratio of about 1:99 w/w % to about 99:1 w/w %.

8. The method of claim 7, wherein the hydroxide bases are in a ratio of about 25:75 w/w % to about 75:25 w/w %.

9. The method of claim 1, wherein the solvent system and the fluorinated material are provided to the batch reactor by a single substance, and no additional solvent is added.

10. The method of claim 1, wherein the fluorinated material, the hydroxide base, and the solvent system are placed in the batch reactor and allowed to react with one another for a time of about 0.5 hours to about 240 hours.

11. The method of claim 10, wherein the fluorinated material, the hydroxide base, and the solvent system are placed in the batch reactor and allowed to react with one another for a time of about 3 hours to about 120 hours.

12. The method of claim 1, wherein the fluorinated material, the hydroxide base, and the solvent system are placed in the batch reactor and allowed to react with one another for a time of about 4 hours to about 60 hours.

13. The method of claim 1, wherein the fluorinated material, the hydroxide base, and the solvent system are placed in the batch reactor and allowed to react with one another for a time of about 4 hours to about 10 hours.

14. The method of claim 1, wherein the heating step includes heating the suspension to a temperature of about 25° C. to about 300° C.

15. The method of claim 1, wherein the heating step includes heating the suspension to a temperature of about 150° C. to about 200° C.

16. The method of claim 1, wherein the hydroxide base is potassium hydroxide; the heating step includes heating to a temperature of about 180° C.; the fluorinated material is PFAS; the PFAS, the hydroxide base, and the solvent system are placed in the batch reactor and allowed to react with one another for a time of about 4 hours to about 8 hours; the PFAS and the solvent system are provided by a single substance; and no additional solvent is added to the batch reactors.

17. The method of claim 1 further including incinerating the defluorinated waste product.

18. A system comprising a batch reactor for chemically destroying and disposing a fluorinated material according to the method of claim 1.

19. The system of claim 18, wherein the fluorinated material is a per- and/or polyfluoroalkyl substance (PFAS).

20. The system of claim 19, wherein the PFAS and optionally the solvent system is provided by an aqueous film-forming foam (AFFF) placed in the batch reactor.
